(19) United States
(12) Patent Application Publication
(10) Pub. No.: US 2026/0031354 A1
(43) Pub. Date: Jan. 29, 2026

(54) ADDITIVE FOR SUPPLEMENTING LITHIUM OR SODIUM, PREPARATION METHOD THEREFOR AND USE THEREOF

(71) Applicant: SVOLT ENERGY TECHNOLOGY CO., LTD., Changzhou, Jiangsu (CN)

(72) Inventors: Qiqi QIAO, Changzhou (CN); Pengfei WANG, Changzhou (CN); Zetao SHI, Changzhou (CN); Feng GUO, Changzhou (CN); Zitan LI, Changzhou (CN); Hongxin YANG, Changzhou (CN)

(73) Assignee: SVOLT ENERGY TECHNOLOGY CO., LTD., Changzhou, Jiangsu (CN)

(21) Appl. No.: 18/995,505

(22) PCT Filed: Apr. 22, 2024
(86) PCT No.: PCT/CN2024/089183

§ 371 Date: Jan. 16, 2025

(30) Foreign Application Priority Data
Apr. 21, 2023 (CN) 202310437795.1

(51) Int. Cl. H01M 4/62; H01M 4/02; H01M 4/36; H01M 4/525

(52) U.S. Cl. CPC H01M 4/62; H01M 4/364; H01M 4/525; H01M 2004/021

(57) ABSTRACT

The present disclosure relates to the field of batteries, and provides an additive for supplementing lithium or sodium, a preparation method therefor and a use thereof. The additive is mainly prepared from Oxalic acid, a salt, and a catalyst; the salt comprises a lithium salt or a sodium salt; the particle size distribution concentration ratio of the additive for supplementing lithium or sodium satisfies the following expression: 1≤(D90−D10)/D50≤100; the specific surface area of the additive for supplementing lithium or sodium is S, and S and D10, D50 and D90 of the additive for supplementing lithium or sodium satisfy the following expression: 1≤(S/((D90−D10)/D50)≤100. The additive provided by the present disclosure is low in decomposition voltage, high in specific capacity, small in particle size, and low in catalyst consumption.

### CROSS REFERENCE TO RELATED APPLICATIONS

The present application claims priority to Chinese Patent Application No. 2023104377951, entitled “ADDITIVE FOR SUPPLEMENTING LITHIUM OR SODIUM, PREPARATION METHOD THEREFOR AND USE THEREOF”, and filed to China National Intellectual Property Administration on Apr. 21, 2023, the entire contents of which are incorporated herein by reference.

### TECHNICAL FIELD

The present disclosure belongs to the field of batteries and relates to an additive for supplementing lithium or sodium and a preparation method and use thereof.

### BACKGROUND

When lithium ion batteries and sodium ion batteries are charged in the first cycle, a SEI film will form on the surface of the negative electrode, causing irreversible capacity loss of the positive electrode material, thereby affecting the capacity and cycle life of the battery. Therefore, the capacity and cycle performance of the battery can be improved by supplementing lithium or sodium at the positive or negative electrode. Compared with supplement lithium and supplement sodium at the negative electrode, supplement lithium and supplement sodium at the positive electrode is simpler and easier. It does not require additional production processes. It only requires adding lithium supplements or sodium supplements in the positive electrode homogenate. Common positive electrode lithium supplements include Li₂O, Li₂O₂, Li₂S, Li₃N, Li₂NiO₂, Li₂CuO₂, Li₂MnO₃, Li₂C₂O₄, Li₅FeO₄, etc. Common positive electrode sodium supplements include Na₂O, Na₂O₂, Na₂S, Na₃N, Na₂NiO₂, Na₂CuO₂, Na₂C₂O₄, Na₅FeO₄, etc. The positive electrode lithium supplements that have begun to be commercialized are mainly Li₂NiO₂ and Li₅FeO₄, and the positive electrode sodium supplements have not yet been commercialized. However, the material stability of Li₂NiO₂ is poor and surface coating is required. The lithium supplement efficiency is low and the product is LiNiO₂, which has poor structural stability and serious gas production at high temperatures. Li₅FeO₄ releases a large amount of oxygen during the first delithiation process, oxidizing the electrolyte and causing gas production in the battery cell. LiOH is easily retained on the surface during the synthesis process, resulting in a homogenous gel jelly. In addition, the synthesis conditions of these two lithium supplements are harsh, and the moisture content in the synthesis process needs to be strictly controlled, resulting in high manufacturing costs. Therefore, it is imperative to develop a low-cost, residue-free, and air-stable lithium and sodium supplement.

Lithium oxalate and sodium oxalate are low-cost, air-stable, acidic lithium and sodium supplements that leave no residue after the first cycle of charging. However, their decomposition voltage is high (4.7V), which does not match the voltage of the current mainstream ternary positive electrode material lithium iron phosphate, and therefore has not been commercialized. In Patent CN114300680A, unmodified lithium oxalate was dissolved in water, then cobalt oxide quantum dot dispersion was slowly dropped into the above solution, and then carbon nanotube dispersion was slowly dropped into the above solution after stirring uniformly to obtain a precursor solution, and then the precursor solution was atomized under the action of an ultrasonic atomizer to finally obtain modified lithium oxalate. The synthesis process of lithium oxalate is relatively complicated. In Patent CN114464909, the prepared catalyst was dispersed in a saturated aqueous solution of lithium oxalate, stirred evenly, ethanol was slowly added to the dispersion, lithium oxalate was precipitated by recrystallization, and then centrifugal drying was carried out to obtain a composite lithium supplement material containing lithium oxalate and the catalyst. In Patent CN110112475A, oxalic acid and sodium carbonate solutions were prepared respectively, then the oxalic acid solution was added slowly to the sodium carbonate solution to form a uniform solution, then the mixed solution was added to ethanol to generate a precipitate, filtered, and dried to obtain the final product of sodium oxalate Na₂C₂O₄, but the sodium oxalate synthesized by this method had a larger particle size of about 1 μm.

### SUMMARY OF THE INVENTION

The present disclosure provides an additive for supplementing lithium or sodium mainly prepared from oxalic acid, a salt and a catalyst;
wherein, the salt comprises: a lithium salt or a sodium salt;
the particle size distribution concentration ratio of the additive for supplementing lithium or sodium satisfies the following formula:

$$1 \leq ( D 90 - D 10 ) / D 50 \leq 100 ;$$

the specific surface area of the additive for supplementing lithium or sodium is S, and S and D10, D50 and D90 of the additive for supplementing lithium or sodium satisfy the following formula:

$$1 \leq ( S / ( ( D 90 - D 10 ) / D 50 ) \leq 100 .$$

In some embodiments, D50 of oxalic acid, the salt, the catalyst and the additive for supplementing lithium or sodium are Da, Db, Dc and Dd respectively, and Da, Db, Dc and Dd satisfy the following formula:

$$0.5 \leq ( Da + Db ) / ( Dc + Dd ) \leq 200 .$$

In some embodiments, a mass ratio of oxalic acid, the salt and the catalyst is (170 to 180):(100 to 220):(5 to 30).

In some embodiments, the additive for supplementing lithium or sodium has a particle size of 0.01 μm to 50 μm.

In some embodiments, the additive for supplementing lithium or sodium has a pH of less than 7.

In some embodiments, the additive for supplementing lithium or sodium has a bulk density of 0.5 g/cm³ to 1.5 g/cm³.

In some embodiments, the catalyst comprises at least one of LiCoO₂, LiNiₓCo_{y}Mn_{1-x-y}O₂, LiFePO₄, LiMnₓFe₁₋ₓO₄, LiMn₂O₄, LiNi_{0.5}Mn_{1.5}O₄, lithium-rich manganese-based positive electrode, NiO, MnO₂, Mn₃O₄, CoO, Co₃O₄, Fe₃O₄, MoO₃, WO₃, Nb₂O₅, Mo₂C, TaC, SiC, TiN, MoN, WN, TiB₂, WB, Ketjen black, conductive carbon super-P, acetylene black, CNT, VGCF, polyaniline, polypyrrole, polythiophene and polypyridine; in the LiNiₓCo_{y}Mn_{1-x-y}O₂, x+y=1, 0<x≤1, 0<y<1; and in the LiMnₓFe₁₋ₓO₄, 0≤x≤1.

In some embodiments, the lithium salt comprises: Li₂CO₃ and/or LiOH.

In some embodiments, the sodium salt comprises at least one of NaOH, Na₂CO₃ or NaHCO₃.

The present disclosure also provides a method for preparing the additive for supplementing lithium or sodium above, wherein the method comprises the following steps:
mixing a mixed solution containing oxalic acid, the salt and the catalyst and then sand milling and spray drying.

In some embodiments, the sand milling is carried out for a time period of 15 to 30 minutes.

In some embodiments, the sand milling has a material filling efficiency of 40% to 80%.

In some embodiments, the spray drying is carried out at a pressure of 0.2 to 1.0 MPa.

The present disclosure also provides a positive electrode material comprising the additive for supplementing lithium or sodium above.

The present disclosure also provides a lithium ion or sodium ion battery comprising the positive electrode material above.

### DESCRIPTION OF THE DRAWINGS

In order to more clearly illustrate the technical solutions of the embodiments of the present disclosure, the drawings required for use in the embodiments will be briefly introduced below. It should be understood that the following drawings only illustrate the embodiments of the present disclosure by way of example, and the dimensional ratios in the drawings do not directly correspond to the actual ratios of the embodiments. At the same time, the following drawings only show certain embodiments of the present disclosure and therefore should not be regarded as limiting the scope.

FIG. 1 is a SEM image (1,000 times) of the additive for supplementing lithium or sodium provided in Example 1 of the present disclosure.

FIG. 2 is a SEM image (20,000 times) of the additive for supplementing lithium or sodium provided in Example 1 of the present disclosure.

FIG. 3 is a graph showing the lithium ion battery charging test results.

FIG. 4 is a graph showing the sodium ion battery charging test results.

### DETAILED DESCRIPTION

Advantages of the embodiments in the content of the invention will be explained in the following embodiment section of the specification, and some of them are obvious from the specification, or can be obtained through some embodiments of the disclosed embodiments.

The technical solution of the present disclosure is further illustrated below with reference to the drawings and some embodiments.

In order to make the objections, technical solutions and advantages of the present disclosure more clearly understood, the present disclosure is further described in detail below in conjunction with the drawings and embodiments. It should be understood that the embodiments described herein are only used to explain the present disclosure, and are not used to limit the present disclosure. In addition, the technical features involved in each embodiment of the present disclosure described below can be combined with each other as long as there is no conflict between them. Without departing from the principles of the embodiments of the present disclosure, several improvements and modifications may be made, and these improvements and modifications are also considered to be within the protection scope of the embodiments of the present disclosure.

The present disclosure provides an additive for supplementing lithium or sodium mainly prepared from oxalic acid, a salt and a catalyst;
wherein, the salt comprises: a lithium salt or a sodium salt;
the particle size distribution concentration ratio of the additive for supplementing lithium or sodium satisfies the following formula:

$$1 \leq ( D 90 - D 10 ) / D 50 \leq 100 ;$$

the specific surface area of the additive for supplementing lithium or sodium is S, and S and D10, D50 and D90 of the additive for supplementing lithium or sodium satisfy the following formula:

$$1 \leq ( S / ( ( D 90 - D 10 ) / D 50 ) \leq 100 .$$

In the additive for supplementing lithium or sodium of in the present disclosure, raw materials are strictly selected and the particle size distribution, specific surface area of the additive and the particle size of the raw materials are strictly designed, so that the additive is low in decomposition voltage, high in specific capacity, small in particle size, and low in catalyst consumption.

The particle size distribution concentration ratio of the additive for supplementing lithium or sodium satisfies the following formula: 1≤(D90−D10)/D50≤100. If the concentration ratio is too high, D90 of the material is too large and there are too many large particles, resulting in less contact with the catalyst and low material capacity. If the concentration ratio is too low, the material particles are too small and side reactions will occur with the electrolyte, resulting in a decrease in battery capacity and a decrease in cycle performance.

The particle size of catalyst is small and the specific surface area is too large, which will easily lead to gelation during the positive electrode homogenization process; the particle size of additive is too small, which is not conducive to the uniform mixing of the additive and the positive electrode material; the specific surface area of the additive is S, and S and D10, D50 and D90 of the additive for supplementing lithium or sodium satisfy the following formula: 1≤(S/((D90−D10)/D50)≤100. If the parameter is too large, it means that the specific surface area is too large and the particle size distribution concentration is small, which is not conducive to the mixing and homogenization of additives and positive electrode materials; if the parameter is too small, it means that the specific surface area is too small, the particle size distribution concentration is large, and the material particle size is large, resulting in low capacity utilization.

In some embodiments, D50 of oxalic acid, the salt, the catalyst and the additive for supplementing lithium or sodium are Da, Db, Dc and Dd respectively, and Da, Db, Dc and Dd satisfy the following formula:

$$0.5 \leq ( Da + Db ) / ( Dc + Dd ) \leq 200 .$$

D50 of oxalic acid, the salt, the catalyst and the additive for supplementing lithium or sodium are Da, Db, Dc and Dd respectively, and Da, Db, Dc and Dd satisfy the following formula: 0.5≤(Da+Db)/(Dc+Dd)≤200. This formula must be satisfied between raw materials and finished products. If this parameter is too low, it indicates that the particle size of oxalic acid and lithium salt used is small, and the particle size of the catalyst and the synthesized additive is too large. On the one hand, it is not conducive to uniform mixing during the sand milling process. On the other hand, the particle size of catalyst is large, the catalytic effect is poor, the particle size of additive is large, the path of lithium ion embedding and disembedding is long, and the material rate performance is poor, resulting in the capacity cannot be fully utilized; if this parameter is too high, it indicates that the particle size of oxalic acid and the salt used is large, and the particle size of the catalyst and the additive is small, which is not conducive to uniform mixing during the sand milling process.

In some embodiments, a mass ratio of oxalic acid, the salt and the catalyst is (170 to 180):(100 to 220):(5 to 30), e.g., 170:220:5, 172:200:10, 174:180:14, 176:160:20, 178:140:25 or 180:100:30.

In some embodiments, the additive for supplementing lithium or sodium has a particle size of 0.01 μm to 50 μm, e.g., 0.01 μm, 0.05 μm, 0.1 μm, 0.5 μm, 1 μm, 5 μm, 10 μm, 15 μm, 20 μm, 25 μm, 30 μm, 35 μm, 40 μm, 45 μm or 50 μm.

In some embodiments, the additive for supplementing lithium or sodium has a pH of less than 7.

In some embodiments, the additive for supplementing lithium or sodium has a bulk density of 0.5 g/cm³ to 1.5 g/cm³, e.g., 0.5 g/cm³, 0.7 g/cm³, 0.9 g/cm³, 1.1 g/cm³, 1.3 g/cm³, or 1.5 g/cm³.

In some embodiments, the catalyst comprises at least one of LiCoO₂, LiNiₓCo_{y}Mn_{1-x-y}O₂, LiFePO₄, LiMnₓFe₁₋ₓO₄, LiMn₂O₄, LiNi_{0.5}Mn_{1.5}O₄, lithium-rich manganese-based positive electrode, NiO, MnO₂, Mn₃O₄, CoO, Co₃O₄, Fe₃O₄, MoO₃, WO₃, Nb₂O₅, Mo₂C, TaC, SiC, TiN, MoN, WN, TiB₂, WB, Ketjen black, conductive carbon super-P, acetylene black, CNT, VGCF, polyaniline, polypyrrole, polythiophene and polypyridine; in the LiNiₓCo_{y}Mn_{1-x-y}O₂, x+y=1, 0<x≤1, 0<y<1; and in the LiMnₓFe₁₋ₓO₄, 0≤x≤1.

In some embodiments, the lithium salt comprises: Li₂CO₃ and/or LiOH.

In some embodiments, the sodium salt comprises at least one of NaOH, Na₂CO₃ or NaHCO₃.

The present disclosure also provides a method for preparing the additive for supplementing lithium or sodium above, wherein the method comprises the following steps:
mixing a mixed solution containing oxalic acid, the salt and the catalyst and then sand milling and spray drying.

The method for preparing the additive for supplementing lithium or sodium of the present disclosure is simple and easy, and has important practical significance for accelerating the commercialization of lithium oxalate and sodium oxalate; the additive for supplementing lithium or sodium with excellent performance can be obtained by sand milling and spray drying oxalic acid, lithium salt (sodium salt) and catalyst; the additives synthesized by the method are all nano-scale, and are mixed very evenly with the catalyst, with extremely low manufacturing cost, and are easy to mass-produce and commercialize.

In some embodiments, the sand milling is carried out for a time period of 15 to 30 minutes, eg, 15 minutes, 20 minutes, 25 minutes, or 30 minutes.

In some embodiments, the sand milling has a material filling efficiency of 40% to 80%, eg, 40%, 45%, 50%, 55%, 60%, 65%, 70%, 75%, or 80%.

In some embodiments, the spray drying is carried out at a pressure of 0.2 to 1.0 MPa, eg, 0.2 MPa, 0.4 MPa, 0.6 MPa, 0.8 MPa, or 1.0 MPa.

In some embodiments, the spray drying is followed by stoving.

In some embodiments, the stoving is carried out at a temperature of 95° C. to 150° C., e.g., 95° C., 100° C., 105° C., 110° C., 115° C., 120° C., 125° C., 130° C., 135° C., 140° C., 145° C., or 150° C.

In some embodiments, the stoving is carried out for a time period of 5 to 15 h, e.g., 5 h, 6 h, 7 h, 8 h, 9 h, 10 h, 11 h, 12 h, 13 h, 14 h, or 15 h.

The present disclosure also relates to a positive electrode material, comprising the additive for supplementing lithium or sodium above.

The present disclosure also relates to a lithium ion or sodium ion battery comprising the positive electrode material above.

### EXAMPLE

The following are typical but non-limiting examples of the present disclosure.

### Example 1

The method for preparing the additive for supplementing lithium provided in this example comprises the following steps:
1. 173 g of oxalic acid dihydrate (H₂C₂O₄·2H₂O), 115 g of lithium hydroxide monohydrate (LiOH·H₂O), 7.8 g of Co₃O₄, and 7.8 g of KB (Ketjen black) were dissolved in 400 g of water and stirred continuously for 4 h;
2. After the reaction was completed, the solution was added to the sand mill for sand milling for 23 minutes, and the material filling efficiency was 60%;
3. The sand-milled material was added into the spray dryer for spraying at a pressure of 0.2 MPa;
4. The sprayed material was dried in an oven at 100° C. for 10 hours to obtain the final product.

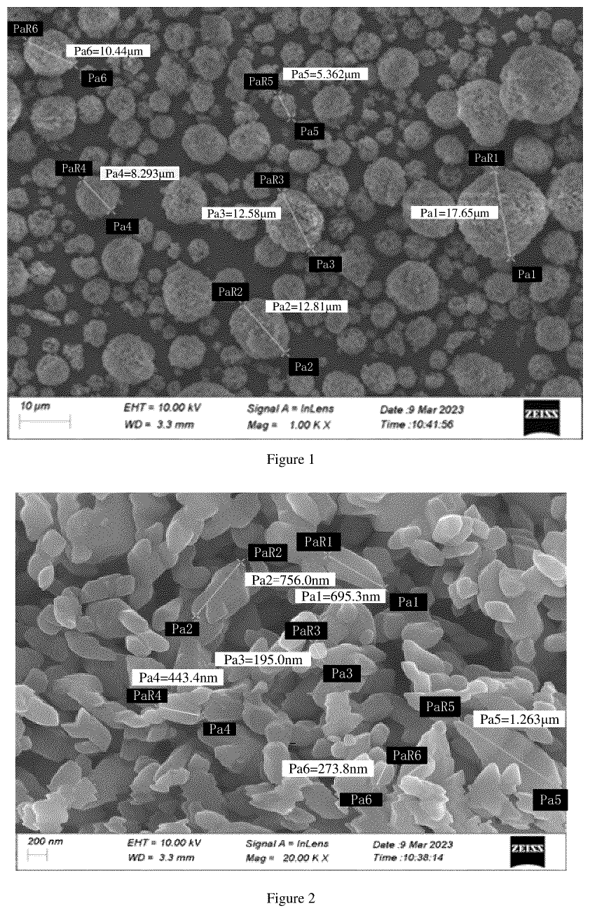

The additive obtained in Example 1 was observed using a scanning electron microscope. FIG. 1 is a low magnification electron microscope image. In FIG. 1, Pa1 is equal to 17.65 μm, Pa2 is equal to 12.81 μm, Pa3 is equal to 12.58 μm, Pa4 is equal to 8.293 μm, Pa5 is equal to 5.362 μm, and Pa6 is equal to 10.44 μm; FIG. 2 is a high magnification electron microscope image. In FIG. 2, Pa1 is equal to 695.3 nm, Pa2 is equal to 756 nm, Pa3 is equal to 195.0 nm, Pa4 is equal to 443.4 nm, Pa5 is equal to 1.263 μm, and Pa6 is equal to 273.8 nm. As can be seen from FIGS. 1 and 2, the additive is spherical in shape, and the primary particle size is about 400 nm, which is a nanomaterial.

### Example 2

The method for preparing the additive for supplementing lithium provided in this example comprises the following steps:
1. 170 g of oxalic acid dihydrate (H₂C₂O₄·2H₂O), 110 g of lithium hydroxide monohydrate (LiOH·H₂O), 5 g of Co₃O₄, and 5 g of KB (Ketjen black) were dissolved in 400 g of water and stirred continuously for 4 h;
2. After the reaction was completed, the solution was added to the sand mill for sand milling for 15 minutes, and the material filling efficiency was 40%;
3. The sand-milled material was added into the spray dryer for spraying at a pressure of 0.2 MPa;
4. The sprayed material was dried in an oven at 100° C. for 10 hours to obtain the final product.

### Example 3

The method for preparing the additive for supplementing lithium provided in this example comprises the following steps:
1. 180 g of oxalic acid dihydrate (H₂C₂O₄·2H₂O), 120 g of lithium hydroxide monohydrate (LiOH·H₂O), 10 g of Co₃O₄, and 10 g of KB (Ketjen black) were dissolved in 400 g of water and stirred continuously for 4 h;
2. After the reaction was completed, the solution was added to the sand mill for sand milling for 30 minutes, and the material filling efficiency was 80%;
3. The sand-milled material was added into the spray dryer for spraying at a pressure of 0.2 MPa;
4. The sprayed material was dried in an oven at 100° C. for 10 hours to obtain the final product.

### Example 4

The method for preparing the additive for supplementing lithium provided in this example comprises the following steps:
1. 176 g of oxalic acid dihydrate (H₂C₂O₄·2H₂O), 118 g of lithium hydroxide monohydrate (LiOH·H₂O), 8.5 g of Co₃O₄, and 8.5 g of KB (Ketjen black) were dissolved in 400 g of water and stirred continuously for 4 h;
2. After the reaction was completed, the solution was added to the sand mill for sand milling for 26 minutes, and the material filling efficiency was 75%;
3. The sand-milled material was added into the spray dryer for spraying at a pressure of 0.2 MPa;
4. The sprayed material was dried in an oven at 100° C. for 10 hours to obtain the final product.

### Example 5

The method for preparing the additive for supplementing lithium provided in this example comprises the following steps:
1. 173 g of oxalic acid dihydrate (H₂C₂O₄·2H₂O), 115 g of lithium hydroxide monohydrate (LiOH·H₂O), 7.8 g of polyaniline, and 7.8 g of LiCoO₂ were dissolved in 400 g of water and stirred continuously for 4 h;
Steps 2 to 4 are the same as in Example 1.

### Example 6

The method for preparing the additive for supplementing lithium provided in this example comprises the following steps:
1. 173 g of oxalic acid dihydrate (H₂C₂O₄·2H₂O), 115 g of lithium hydroxide monohydrate (LiOH·H₂O), 7.8 g of WN, and 7.8 g of Fe₃O₄ were dissolved in 400 g of water and stirred continuously for 4 h;
Steps 2 to 4 are the same as in Example 1.

### Example 7

The method for preparing the additive for supplementing sodium provided in this example comprises the following steps:
1. 173 g of oxalic acid dihydrate (H₂C₂O₄·2H₂O), 110 g of NaOH, 7.8 g of Co₃O₄, and 7.8 g of KB (Ketjen black) were dissolved in 400 g of water and stirred continuously for 4 h;
Steps 2 to 4 are the same as in Example 1.

### Comparative Example 1

The only difference from Example 1 is that step 2 and step 3 are omitted.

### Comparative Example 2

The only difference from Example 7 is that step 2 and step 3 are omitted.

### Experimental Example 1

The particle size distribution, specific surface area and pH of the obtained additive were tested using a laser particle size analyzer and a pH meter, and the results are shown in Tables 1 and 2. The D50 of the additive in the examples is between 5.25 μm and 8.38 μm, the pH is between 6.54 and 6.85, and the material is acidic, so it will not affect the homogenization of the positive electrode material. Specific surface area is from 37 m²/g to 57 m²/g. The additives in the comparative examples were not subjected to sand milling and spraying, the particle size of the material did not meet the conditions defined in the present disclosure, and the specific surface area is small.

TABLE 1

| | Particle size (μm) | | | | | |
|---|---|---|---|---|---|---|
| | D50 | D0 | D10 | D90 | D99 | D100 |
| Example 1 | 6.97 | 0.52 | 1.93 | 15.2 | 24.8 | 35.2 |
| Example 2 | 7.25 | 0.68 | 2.13 | 17.9 | 26.5 | 38.2 |
| Example 3 | 6.21 | 0.43 | 1.78 | 14.1 | 22.6 | 32.9 |
| Example 4 | 6.55 | 0.47 | 1.85 | 14.6 | 23.4 | 33.6 |
| Example 5 | 8.38 | 0.75 | 2.25 | 19.5 | 27.8 | 39.7 |
| Example 6 | 7.39 | 0.72 | 2.18 | 18.6 | 27.3 | 38.9 |
| Example 7 | 5.25 | 0.36 | 1.67 | 13.2 | 21.0 | 31.5 |
| Comparative | 60.43 | 12.78 | 28.75 | 86.94 | 120.85 | 150.67 |
| example 1 | | | | | | |
| Comparative | 75.90 | 22.35 | 45.12 | 100.29 | 168.92 | 197.83 |
| example 2 | | | | | | |

TABLE 2

| | Specific surface area (m²/g) | pH |
|---|---|---|
| Example 1 | 50 | 6.78 |
| Example 2 | 41 | 6.54 |
| Example 3 | 55 | 6.65 |
| Example 4 | 52 | 6.71 |
| Example 5 | 37 | 6.75 |
| Example 6 | 39 | 6.63 |
| Example 7 | 57 | 6.85 |
| Comparative example 1 | 0.78 | 6.75 |
| Comparative example 2 | 0.35 | 6.83 |

### Experimental Example 2

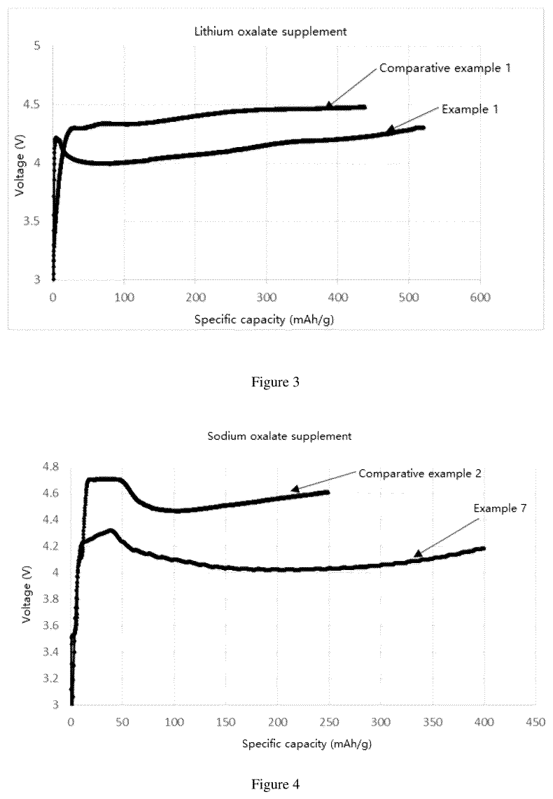

The additives obtained in Examples 1-6, Comparative Example 1 and Comparative Example 3 were respectively mixed with a conductive agent SP (carbon black) and a binder PVDF (polyvinylidene fluoride) in a mass ratio of 8:1:1 to form a positive electrode slurry, which was coated on an aluminum foil to form a positive electrode plate. Metal lithium was used as the negative electrode plate, Celgard 2400 microporous polypropylene membrane was used as the separator, and LiPF₆ (lithium hexafluorophosphate)/EC (ethylene carbonate)-DMC (dimethyl carbonate) was used as the electrolyte to assemble a lithium ion battery. The assembled lithium ion batteries were charged and tested respectively, and the charging test results were shown in FIG. 3.

The additives obtained in Example 7 and Comparative Example 2 were respectively mixed with a conductive agent SP (carbon black) and a binder PVDF (polyvinylidene fluoride) in a mass ratio of 8:1:1 to form a positive electrode slurry, which was coated on an aluminum foil to form a positive electrode plate. Metallic sodium was used as the negative electrode plate, Celgard 2400 microporous polypropylene membrane was used as the separator, and NaPF₆ (sodium hexafluorophosphate)/EC (ethylene carbonate)-DMC (dimethyl carbonate) was used as the electrolyte to assemble a sodium ion battery. The assembled sodium ion batteries were charged and tested respectively, and the charging test results are shown in FIG. 4.

The lithium oxalate prepared in Example 1 has a low decomposition voltage of 4.2V to 4.3V and a specific capacity of 520 mAh/g. The lithium oxalate prepared in Comparative Example 1 has a high decomposition voltage of 4.3V to 4.5V and a specific capacity of 438 mAh/g. The reason of Example 1 has excellent performance is that the material is nanometer-scale, and the lithium oxalate and the catalyst are fully and evenly mixed, which is beneficial for the catalyst to reduce the decomposition voltage of lithium oxalate and improve the capacity.

The sodium oxalate prepared in Example 7 has a low decomposition voltage of 4.2V to 4.3V and a specific capacity of 400 mAh/g. The sodium oxalate prepared in Comparative Example 2 has a high decomposition voltage of 4.4V to 4.7V and a specific capacity of 249 mAh/g. The reason of Example 2 has excellent performance is that the material is nanometer-scale, and the sodium oxalate and the catalyst are fully and evenly mixed, which is beneficial for the catalyst to reduce the decomposition voltage of the sodium oxalate and improve the capacity.

### INDUSTRIAL PRACTICALITY

In summary, the present disclosure provides an additive for supplementing lithium or sodium and a preparation method and use thereof. The additive for supplementing lithium or sodium is low in decomposition voltage, high in specific capacity, small in particle size, and low in catalyst consumption. The preparation method is simple and easy to operate, and the obtained lithium or sodium supplement additive has excellent performance. The additives synthesized by the method are all nano-scale, and are mixed very evenly with the catalyst, with extremely low manufacturing cost, and are easy to mass-produce.

1. An additive for supplementing lithium or sodium, wherein the additive for supplementing lithium or sodium is mainly prepared from oxalic acid, a salt and a catalyst;
wherein the salt comprises: a lithium salt or a sodium salt;
the particle size distribution concentration ratio of the additive for supplementing lithium or sodium satisfies the following formula: 1≤(D90−D10)/D50≤100;
the specific surface area of the additive for supplementing lithium or sodium is S, and S and D10, D50 and D90 of the additive for supplementing lithium or sodium satisfy the following formula:
$$1 \leq ( S / ( ( D 90 - D 10 ) / D 50 ) \leq 100 .$$

2. The additive for supplementing lithium or sodium according to claim 1, wherein D50 of oxalic acid, the salt, the catalyst and the additive for supplementing lithium or sodium are Da, Db, Dc and Dd respectively, and Da, Db, Dc and Dd satisfy the following formula:
$$0.5 \leq ( Da + Db ) / ( Dc + Dd ) \leq 200 .$$

3. The additive for supplementing lithium or sodium according to claim 1, wherein a mass ratio of oxalic acid, the salt and the catalyst is (170 to 180):(100 to 220):(5 to 30).

4. The additive for supplementing lithium or sodium according to claim 1, wherein the additive for supplementing lithium or sodium has a particle size of 0.01 μm to 50 μm.

5. The additive for supplementing lithium or sodium according to claim 1, wherein the additive for supplementing lithium or sodium has a pH of less than 7.

6. The additive for supplementing lithium or sodium according to claim 1, wherein the additive for supplementing lithium or sodium has a bulk density of 0.5 g/cm³ to 1.5 g/cm³.

7. The additive for supplementing lithium or sodium according to claim 1, wherein the catalyst comprises at least one of: LiCoO₂, LiNiₓCo_{y}Mn_{1-x-y}O₂, LiFePO₄, LiMnₓFe₁₋ₓO₄, LiMn₂O₄, LiNi_{0.5}Mn_{1.5}O₄, lithium-rich manganese-based positive electrode, NiO, MnO₂, Mn₃O₄, CoO, Co₃O₄, Fe₃O₄, MoO₃, WO₃, Nb₂O₅, Mo₂C, TaC, SiC, TiN, MoN, WN, TiB₂, WB, Ketjen black, conductive carbon super-P, acetylene black, CNT, VGCF, polyaniline, polypyrrole, polythiophene and polypyridine; in the LiNiₓCo_{y}Mn_{1-x-y}O₂, x+y=1, 0<x≤1, 0<y<1; and in the LiMnₓFe₁₋ₓO₄, 0≤x≤1.

8. The additive for supplementing lithium or sodium according to claim 1, wherein the lithium salt comprises: Li₂CO₃ and/or LiOH.

9. The additive for supplementing lithium or sodium according to claim 1, wherein the sodium salt comprises at least one of NaOH, Na₂CO₃ or NaHCO₃.

10. A method for preparing the additive for supplementing lithium or sodium according to claim 1, wherein the method comprises the following steps:
mixing a mixed solution containing oxalic acid, the salt and the catalyst and then sand milling and spray drying.

11. The method for preparing the additive for supplementing lithium or sodium according to claim 10, wherein the sand milling is carried out for a time period of 15 to 30 minutes.

12. The method for preparing the additive for supplementing lithium or sodium according to claim 10, wherein the sand milling has a material filling efficiency of 40% to 80%.

13. The method for preparing the additive for supplementing lithium or sodium according to claim 10, wherein the spray drying is carried out at a pressure of 0.2 to 1.0 MPa.

14. A positive electrode material, wherein the positive electrode material comprises the additive for supplementing lithium or sodium according to claim 1.

15. A lithium ion or sodium ion battery, wherein the lithium ion or sodium ion battery comprises the positive electrode material according to claim 14.
